(19) United States
(12) Patent Application Publication
(10) Pub. No.: US 2026/0266698 A1
(43) Pub. Date: Sep. 10, 2026

(54) FORENSIC SAMPLE COLLECTION DEVICES, KITS, AND METHODS

(71) Applicants: EASTERN KENTUCKY UNIVERSITY, Richmond, KY (US); KENTUCKY COMMUNITY AND TECHNICAL COLLEGE SYSTEM, Versailles, KY (US)

(72) Inventors: Jamie D. Fredericks, Lexington, KY (US); Eric N. Wooldridge, Stanford, KY (US)

(21) Appl. No.: 19/148,620

(22) PCT Filed: Jan. 17, 2024
(86) PCT No.: PCT/US2024/011719

§ 371 Date: Jul. 16, 2025

Related U.S. Application Data
(60) Provisional application No. 63/439,304, filed on Jan. 17, 2023.

(51) Int. Cl. G01N 1/10

(52) U.S. Cl. CPC G01N 1/10

(57) ABSTRACT

A sample collection device includes a handle and a head. The head receives and maintains a sample card for collecting one or more forensic samples at a distal end of the sample collection device and in a manner which permits the sample card to be placed into contact with physical evidence. The head can include a platform and a lid. The platform includes a proximal end secured to the handle and a distal end that defines an upper surface for receiving the sample card. The lid is moveable relative to the platform between a first (closed) position to maintain the sample card in position on the upper surface of the platform while the sample collection device is in use and a second (open) position to permit deposit or removal of the sample card. A kit for collecting forensic evidence and method for collecting and processing forensic evidence are also provided.

### RELATED APPLICATION

The present application claims priority to U.S. Patent Application Ser. No. 63/439,304, filed on Jan. 17, 2023, the entire disclosure of which is incorporated herein by reference.

### TECHNICAL FIELD

The presently disclosed subject matter relates to devices, kits, and methods for forensic sample collection.

### BACKGROUND

Genetic testing has become a vital source of evidence in modern criminal investigations. Current practices use Polymerase Chain Reaction (PCR) testing to obtain genetic evidence. Traditional PCR involves the steps of: collection, extraction, quantitation, amplification, profiling, and analysis (FIG. 1). This process is time-consuming mostly due to the extraction and quantitation steps. Direct PCR is a method that increases the efficiency within these steps to improve testing time. Direct PCR has been available for testing with reference samples, such as suspects being swabbed directly or blood collected on a storage card.

However, forensic or crime scene evidence still uses the traditional PCR techniques because of the problematic nature of collection at a crime scene. For example, crime scene evidence can be in hard-to-reach locations and/or on porous or easily degradable surfaces, such as rocks and soil. These locations are also susceptible to degradation from exposure to natural weather conditions and human interference. For these reasons, collecting the samples as soon as possible is a priority for crime scene investigators. Conventionally, investigators rely on traditional swabs to reach these surfaces and allow for preservation when transporting back to the crime lab. But conventional swabs limit the number of times the sample can be tested. If testing produces an inadequate result or if new information leads to a need for further testing, any swab that has been used in testing cannot undergo further testing.

Accordingly, there remains a need for new approaches for collecting forensic samples which facilitate testing of such samples using direct PCR. As such, novel devices, kits, and methods for collecting forensic samples would thus be both beneficial and desirable.

### SUMMARY

The presently disclosed subject matter meets some or all of the above-identified needs, as will become evident to those of ordinary skill in the art after a study of information in this document.

This Summary describes several embodiments of the presently disclosed subject matter and, in many cases, lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features or all embodiments disclosed herein.

An exemplary sample collection device made in accordance with the present disclosure includes: a handle; and a head secured to a distal end of the handle. The head is configured to receive and maintain a sample card for collecting one or more forensic samples at a distal end of the sample collection device and in a manner which permits the sample card to be placed into contact with physical evidence. The head can include a platform and a lid. The platform includes a proximal end that is secured to the distal end of the handle and a distal end that defines an upper surface for receiving the sample card. The lid is moveable relative to the platform, such that the lid can be transitioned between a first (closed) position and a second (open) position. When the lid is in the closed position and the storage card is placed on the upper surface of the platform, the lid sandwiches the sample card between the lid and the platform. Conversely, when the lid is in the open position, the upper surface of the platform is sufficiently uncovered as to permit deposit of the sample card onto, or removal of the sample card from, the upper surface of the platform. In instances where biological samples including deoxyribonucleic acid (DNA) are collected, the sample card will ordinarily preserve the collected samples in a manner which enables testing of the samples using direct PCR.

In some embodiments, the lid is configured to receive at least a portion of the platform when the lid is placed in the closed position. In some embodiments, the lid includes a cavity in which the distal end of the platform is deposited when the lid is placed in the closed position. In some embodiments, the lid includes an opening. In some embodiments, the opening of the lid corresponds to a central opening of the lid through which a portion of the platform extends when the lid is in the closed position. In some embodiments, the lid includes a permeable material that spans across the opening of the lid. In some embodiments, the permeable material is a mesh.

In some embodiments, the lid is removably secured to the platform, such that the lid can be completely removed from engagement with the platform to transition the lid to the open position. In some embodiments, the lid is pivotally mounted to the platform. In some embodiments, the lid is maintained in the closed position by a latched-based connection. In one embodiment, one of the platform and the lid includes one or more latches and the other of the platform and the lid includes one or more notches. When the lid is placed in the closed position at the one or more latches are at least partially received in the one or more notches. In some embodiments, the lid is maintained in the closed position by a friction fit connection when the head is loaded with the sample card and the lid is placed in the closed position.

In some embodiments, the platform includes a reservoir that is configured to receive a liquid, such as a buffer solution. In some embodiments, the reservoir is in fluid communication with an opening defined by the upper surface of the platform to facilitate selective wetting of the sample card. In some embodiments, the reservoir is in fluid communication with an opening defined by an exterior side surface of the platform to facilitate loading of the reservoir with a liquid.

In some embodiments, the upper surface of the platform is a planar surface. In some embodiments, the upper surface of the platform forms an angle with respect to a longitudinal axis of the platform. In some embodiments, the upper surface forms an angle between about 20 degrees to about 60 degrees with respect to the longitudinal axis of the platform.

In some embodiments, the platform is removably secured to the handle. In one embodiment, one of the proximal end of the platform and a distal end of the handle includes a threaded body and the other of the proximal end of the platform and the distal end of the handle includes a threaded opening for receiving the threaded body. In some embodiments, the handle includes a plurality of depressions and a plurality of protrusions that provide gripping locations along the length of the handle. In some embodiments, the handle is symmetrical about a longitudinal axis of the handle.

An exemplary kit for collecting a forensic evidence includes: a sample card configured to collect one or more samples when placed into contact with physical evidence; and a sample collection device. The sample collection device include a handle and a head that is secured to a distal end of the handle and is configured to receive and maintain the sample card at a distal end of the of the sample collection device. The head can include a platform and a lid. The platform includes a proximal end that is secured to the handle, and a distal end that defines an upper surface of the platform for receiving the sample card. The lid is moveable relative to the platform. In some embodiments, the kit further includes a buffer solution.

An exemplary method for collecting and processing forensic evidence includes loading a sample card for collecting one or more samples onto an upper surface of a platform of a sample collection device. The sample card is secured into position on the upper surface of the platform by sandwiching the sample card between the platform and a lid of the sample collection device. The sample collection device is positioned so that the sample card is placed into contact with a biological material to collect one or more biological samples. At least one of the one or more collected biological samples to a direct polymerase chain reaction (PCR) protocol.

Further features and advantages of the presently disclosed subject matter will become evident to those of ordinary skill in the art after a study of the description, figures, and non-limiting examples in this document.

### BRIEF DESCRIPTION OF THE DRAWINGS

FIG. 1 is a diagram showing steps involved in traditional PCR and direct PCR.

FIG. 2 is a perspective view of an exemplary sample collection device made in accordance with the present disclosure.

FIG. 3 is a side view of the exemplary sample collection device of FIG. 2.

FIG. 4 is a partial exploded view of the exemplary sample collection device of FIG. 2.

FIG. 5 is an exploded view of a head of the exemplary sample collection device of FIG. 2, with a lid of the head of the exemplary sample section collection device shown in a sectional view to better illustrate certain features thereof.

FIG. 6A is a side view of another head which can be utilized in an exemplary sample collection device made in accordance with the present disclosure, with a lid of the head in a first position.

FIG. 6B is another side view of the head of FIG. 6A, but with the lid of the head in a second position.

FIG. 7 is a side view of another head which can be utilized in an exemplary sample collection device made in accordance with the present disclosure.

FIG. 8 is a side view of another head which can be utilized in an exemplary sample collection device made in accordance with the present disclosure.

FIG. 8A is a sectional view of the head of FIG. 8.

FIG. 9 is a perspective view of another head which can be utilized in an exemplary sample collection device made in accordance with the present disclosure.

FIG. 9A is a sectional view of the head of FIG. 9.

FIG. 10 is a side view of another head which can be utilized in an exemplary sample collection device made in accordance with the present disclosure.

FIG. 10A is a sectional view of the head of FIG. 10.

FIG. 11 is a perspective view of another head which can be utilized in an exemplary sample collection device made in accordance with the present disclosure.

FIG. 12 is a sectional view of the head of FIG. 11.

FIG. 13 is a side schematic view of a platform which can be utilized in an exemplary sample collection device made in accordance with the present disclosure.

FIG. 14 is a side schematic view of another platform which can be utilized in an exemplary sample collection device made in accordance with the present disclosure.

FIG. 15 is a perspective view of another platform which can be utilized in an exemplary sample collection device made in accordance with the present disclosure.

FIG. 16 is a perspective view of another platform which can be utilized in an exemplary sample collection device made in accordance with the present disclosure.

FIG. 17 is a perspective view of another platform which can be utilized in an exemplary sample collection device made in accordance with the present disclosure.

FIG. 18 is a perspective view of another platform which can be utilized in an exemplary sample collection device made in accordance with the present disclosure.

FIG. 19 is a perspective view of another platform which can be utilized in an exemplary sample collection device made in accordance with the present disclosure.

FIG. 20 is a top view of a permeable material which can be utilized in an exemplary sample collection device made in accordance with the present disclosure.

FIG. 21 is a top view of another permeable material which can be utilized in an exemplary sample collection device made in accordance with the present disclosure.

FIG. 22A is a chart showing a comparison of the deoxyribonucleic acid (DNA) concentration within samples obtained by a nylon swab, the exemplary sample collection device of FIG. 2 loaded with a Whatman® Fast Technology for Analysis of nucleic acids (FTA) classic card, and the exemplary collection device of FIG. 2 loaded with a Copan® Nucleic-Card from a cotton material with 10 μl of blood deposited thereon. DNA quantified using Promega® Plexor® system.

FIG. 22B is a chart showing a comparison of the deoxyribonucleic acid (DNA) concentration within samples obtained by a nylon swab, the exemplary sample collection device of FIG. 2 loaded with a Whatman® Fast Technology for Analysis of nucleic acids (FTA) classic card, and the exemplary collection device of FIG. 2 loaded with a Copan® Nucleic-Card from a jean material with 10 μl of blood deposited thereon. DNA quantified using Promega® Plexor® system.

FIG. 22C is a chart showing a comparison of the deoxyribonucleic acid (DNA) concentration within samples obtained by a nylon swab, the exemplary sample collection device of FIG. 2 loaded with a Whatman® Fast Technology for Analysis of nucleic acids (FTA) classic card, and the exemplary collection device of FIG. 2 while carrying a Copan® Nucleic-Card from a cotton material with 10 μl of saliva deposited thereon. DNA quantified using Promega® Plexor® system

FIG. 23 is a flow diagram of an exemplary method for collecting and processing forensic evidence.

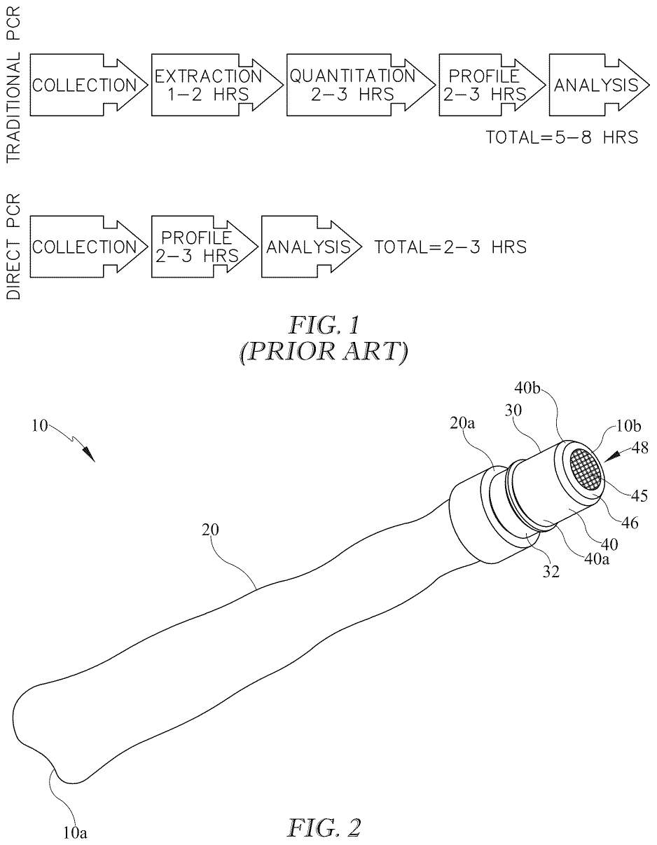

### DESCRIPTION OF EXEMPLARY EMBODIMENTS

The details of one or more embodiments of the presently-disclosed subject matter are set forth in this document. The information provided in this document, and particularly the specific details of the described exemplary embodiments, is provided primarily for clearness of understanding and no unnecessary limitations are to be understood therefrom.

Additionally, while the terms used herein are believed to be well understood by one of ordinary skill in the art, definitions are set forth to facilitate explanation of the presently-disclosed subject matter. Unless defined otherwise, all technical and scientific terms used herein have the same meaning as commonly understood by one of ordinary skill in the art to which the presently disclosed subject matter belongs. Although many methods, devices, and materials similar or equivalent to those described herein can be used in the practice or testing of the presently disclosed subject matter, representative methods, devices, and materials are now described.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Furthermore, following long-standing patent law convention, the terms “a”, “an”, and “the” refer to “one or more” when used in this application, including the claims. Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, length, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Wherever any of the phrases “for example,” “such as,” “including” and the like are used herein, the phrase “and without limitation” is understood to follow unless explicitly stated otherwise. Similarly “an example,” “exemplary” and the like are understood to be non-limiting.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, “biological sample” means a sample of biological material derived from a biological organism. In some embodiments, the biological organism is an animal. In some embodiments the biological organism is a human. In some embodiments, the biological organism is a plant. In some embodiments, the biological material includes deoxyribonucleic acid (DNA) of the biological organism from which the biological material is derived.

All patents, patent applications, published applications and publications, GenBank sequences, databases, websites and other published materials referred to in the present disclosure are, unless noted otherwise, incorporated by reference in their entirety.

The presently disclosed subject matter includes sample collection devices for collecting forensic samples.

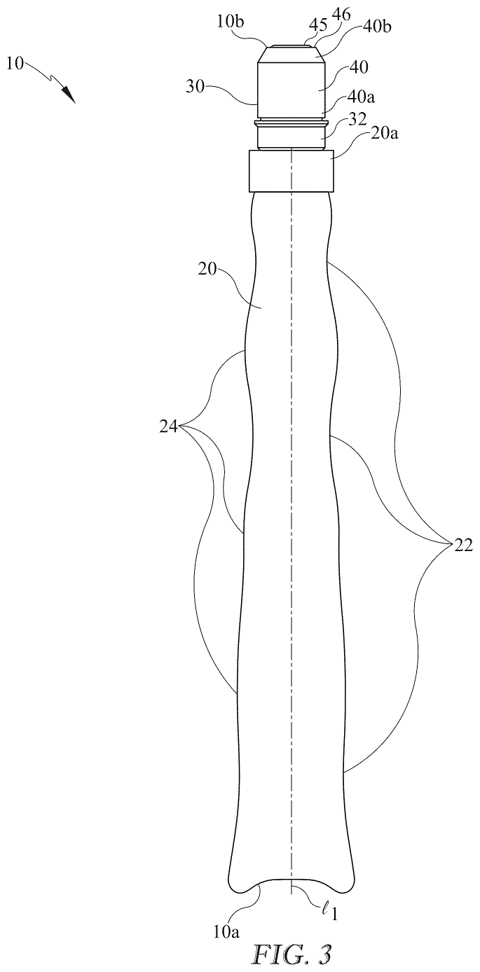

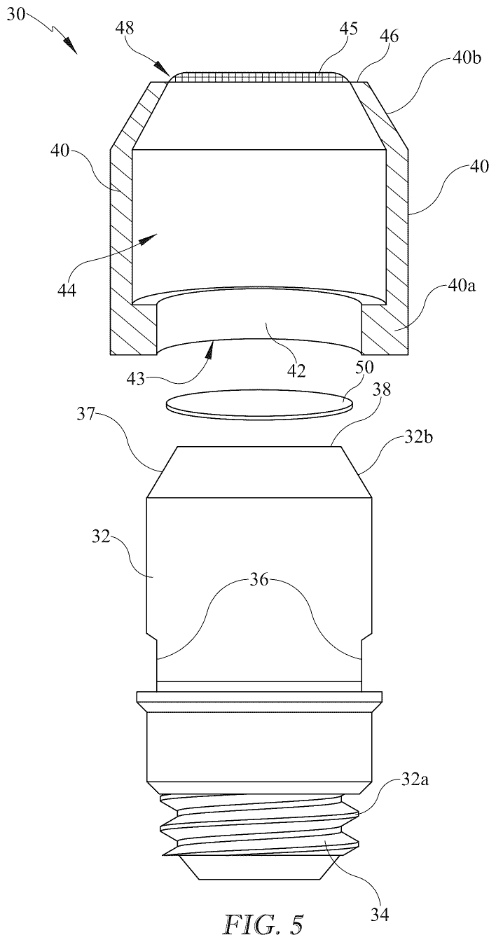

Referring first to FIGS. 2-4, an exemplary sample collection device 10 generally includes: a handle 20; and a head 30 secured to a distal end 20a of the handle 20. The head 30 is configured to receive and maintain a sample card 50 (FIG. 5) for collecting one or more forensic samples at a distal end 10b of the sample collection device 10 and in a manner which permits the sample card 50 to be placed in contact with physical evidence. When the sample collection device 10 is in use and provided with a sample card 50 (which may also be referred to as a storage card or a sample storage card), one or more forensic samples can thus be collected by positioning the distal end 10b of the sample collection device 10 in a manner which places the sample card 50 into direct contact with physical evidence. In instances where biological samples including deoxyribonucleic acid (DNA) are collected, the sample card 50 will ordinarily preserve the collected samples in a manner which enables testing of the samples using direct PCR.

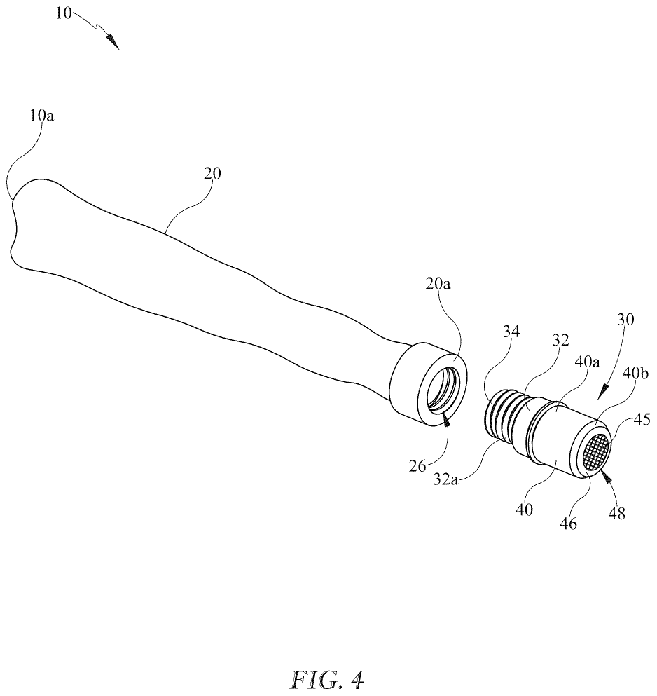

Referring still to FIGS. 2-4, the proximal end 10a of the sample collection device 10 is defined by the proximal end of the handle 20. To facilitate the collection of physical evidence in hard-to-reach locations, in this exemplary embodiment, the handle 20 is in the form of an elongated shaft, with a length of about 12 centimeters (12 cm) to about 13 centimeters (13 cm). Of course, the length of the handle 20 can be adjusted to better accommodate different types of physical evidence and/or environments in which physical evidence is located. In addition to facilitating the collection of physical evidence in hard-to-reach locations, the elongated construction of the handle 20 can also serve to reduce the risk of contaminating physical evidence by increasing the distance between a user collecting the forensic sample and physical evidence. As best shown in FIG. 3, in this exemplary embodiment, the exterior of the handle 20 defines, and thus can be characterized as including, a plurality of depressions 22 and a plurality of protrusions 24 that provide a number of potential gripping locations along the length of the handle 20. As compared to the handle design of traditional swabs, which are generally only capable of applying a vertical pressure of about 0.7 pounds per square inch (PSI), the handle 20 of the sample collection device 10 permits significantly greater vertical pressure to be applied without failure. Specifically, in this exemplary embodiment, the handle is comprised of a rigid, plastic material which permits about ten PSI (10 PSI) to about 15 PSI to be applied without failure. In this exemplary embodiment, the handle 20 is constructed of polylactic acid (PLA) plastic. Of course, alternative materials may be utilized to construct the handle 20 and accommodate different types of physical evidence and/or environments in which physical evidence is located and permit increased or decreased vertical pressure to be applied. For instance, in some embodiments, the handle 20 may be constructed of polypropylene or nylon. Without wishing to be bound by any particular theory, the plurality of depressions 22 and the plurality of protrusions 24 are believed to provide a more ergonomic handle arrangement than the cylindrical handles typical in evidence swabs of known construction. As further shown in FIG. 3, in this exemplary embodiment, the handle 20 is generally symmetrical about a longitudinal axis, l₁, of the handle 20. Accordingly, the handle 20 can readily be gripped by left-handed users and right-handed users alike. As shown in FIG. 4, the distal end 20a of the handle 20 defines a threaded opening 26, the importance of which is further discussed below.

Referring now to FIGS. 2-5, the head 30 of the sample collection device 10 includes: a platform 32 with a proximal end 32a and a distal end 32b; and a lid 40. To better illustrate certain features present in the interior of the lid 40, in FIG. 5, the lid 40 is shown in a sectional view. The exterior of the lid 40 which would be shown absent such sectional view, is consistent with that shown in FIG. 3. The proximal end 32a of the platform 32 is secured to a distal end 20a of the handle 20, and the distal end 32b of the platform 32 defines an upper surface 38 of the platform 32 upon which the sample card 50 can be placed. In some embodiments, the diameter of the upper surface 38 of the platform 32 is between about five millimeters (5 mm) to about ten millimeters (10 mm). In this embodiment, the upper surface 38 is a planar surface. Of course, the shape and/or diameter of the upper surface 38 can be modified to accommodate different types of physical evidence and/or environments in which physical evidence is located.

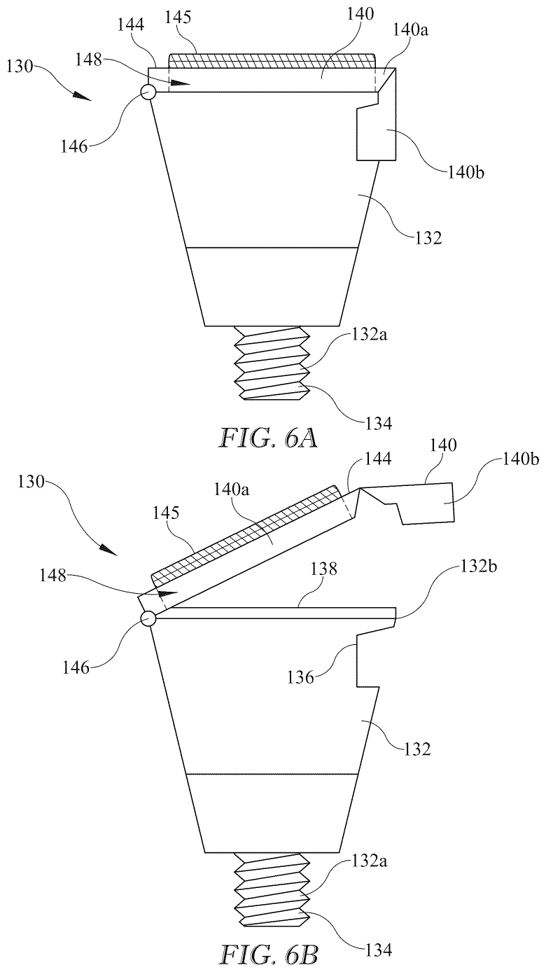

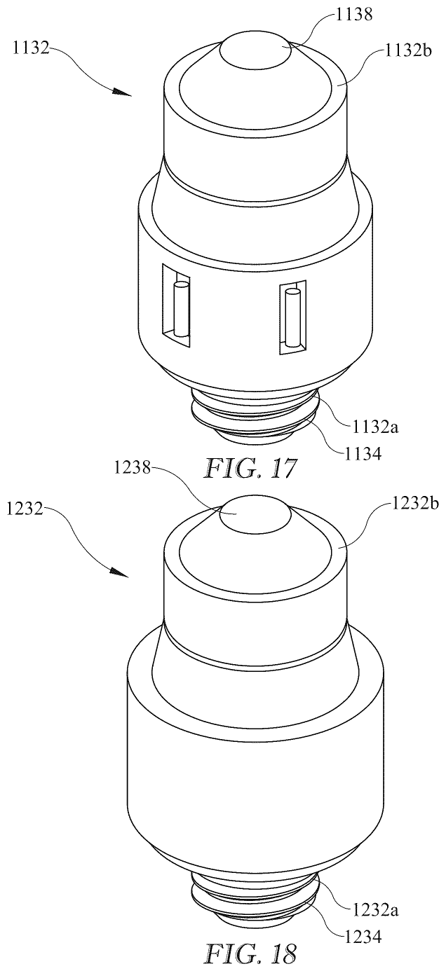

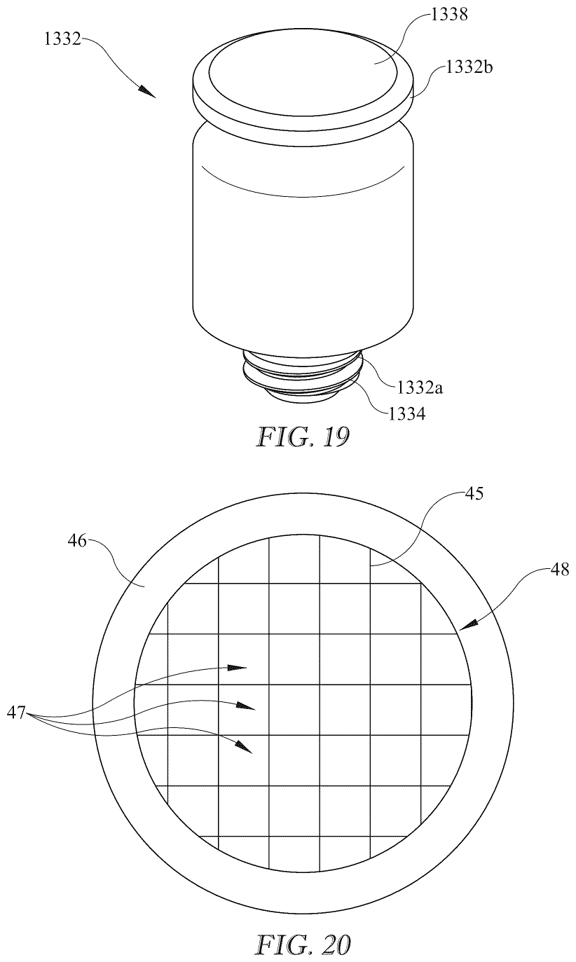

Referring still to FIGS. 2-5, in this exemplary embodiment, the platform 32 is removably secured to the handle 20 via threaded connection between the handle 20 and the platform 32. In this regard, the proximal end 32a of the platform defines, and thus can be characterized as including, a threaded body 34 that is configured to be received in, and mate with the threading of, the threaded opening 26 defined by the distal end 20a of the handle 20. Accordingly, the platform 32, and thus head 30 as a whole when fully assembled, and the handle 20 can be readily associated by inserting the threaded body 34 of the platform 32 in the threaded opening 26 of the handle 20 and rotating the platform 32 in a first rotational direction (e.g., clockwise), and, subsequently, readily disassociated by rotating the platform 32 in a second, opposite rotational direction (e.g., counterclockwise). Of course, alternative means for removably securing the platform 32 to the handle 20 may be employed without departing from the spirit and scope of the present disclosure. Furthermore, while it is generally preferred the handle 20 and the platform be removably secured to each other to facilitate separate storage of the handle 20 and platform 32 and/or simple replacement or substitution of the platform 32, such as with the other exemplary platforms described herein with reference to FIGS. 6A, 6B, 7, 8, 8A, 9, 9A, 10, 10A, and 11-19, or head as a whole, alternative embodiments in which the handle 20 and the platform are permanently secured together are also contemplated herein.

Referring still to FIGS. 2-5, the lid 40 is moveable relative to the platform 32, such that the lid 40 can be transitioned between a first (closed) position (FIGS. 2-4) and a second (open) position (FIG. 5). When the lid 40 is in the closed position and the storage card 50 is placed on the upper surface 38 of the platform 32, the lid 40 sandwiches the sample card 50 between the lid 40 and the platform 32. Specifically, in this embodiment, the lid 40 sandwiches the sample card 50 between lid 40 and the upper surface 38 of the platform 32. Conversely, when the lid 40 is in the open position, the upper surface 38 of the platform 32 is sufficiently uncovered as to permit deposit of the sample card 50 onto, or removal of the sample card 50 from, the upper surface 38. As evidenced in the discussion that follows, embodiments in which the lid of the sample collection device can be completely detached from the platform of the sample collection device, as well embodiments in which the lid can only be partially detached from the platform of the sample collection device, are contemplated herein.

Referring now specifically to FIG. 5, to maintain the lid 40 in the closed position while the sample collection device 10 is in use, the lid 40 and the platform 32 are configured to interlock with each other. As shown, in this exemplary embodiment, the lid 40 and the platform 32 can be selectively interlocked with each other via a latch-based connection. In this regard, the lid 40 includes a latch 42, and the platform 32 includes a corresponding notch 36 that is sized to at least partially receive the latch 42 therein. In this exemplary embodiment, both the latch 42 and the notch 36 are annularly shaped, with the latch 42 being defined by an interior surface of the lid 40 located at a proximal end 40a of the lid 40, and the notch 36 being located between the proximal end 32a and the distal end 32b of the platform 32. Accordingly, to transition the lid 40 to the closed position and interlock the lid 40 and the platform 32 together, the distal end 32b of the platform 32 is inserted through an opening 43 and into an internal cavity 44 defined by the lid 40 until the latch 42 is deposited in the notch 36. In this regard, and in this exemplary embodiment, the lid 40 is configured to receive the platform 32, such that the distal end 32b of the platform 32 is deposited in the internal cavity 44 when the lid 40 is placed in the closed position. To transition the lid 40 to the open position, a user can pull on the lid 40 while holding the handle 20 until the latch 42 is released from the notch 36 and the platform 32 and the lid 40 and the platform 32 are completely separated. Accordingly, in this exemplary embodiment, the lid 40 is fully detached from the platform 32 when in the open position.

Referring now again to FIGS. 2-5, to accommodate insertion and removal of the platform 32 from the internal cavity 44 of the lid 40, the lid 40 is preferably constructed of a material, such as a plastic material, which permits some degree of flexure as the platform 32 is inserted into or removed from the internal cavity 44. Further in this regard, in this exemplary embodiment, an exterior surface 37 defined by the distal end 32b of the platform and located directly beneath the upper surface 38 is sloped, such that, when the platform 32 is inserted in the internal cavity 44, the lid 40 gradually radially expands until the distal end 32b of the platform 32 is fully received in the internal cavity 44. The distal end 32b of the platform 32 may thus be characterized as retaining a wedge-like shape.

Although the lid 40 and the platform 32 are primarily referred to herein as including the latch 42 and the notch 36, respectively, it is appreciated that the locations of the latch 42 and the notch 36 can, in alternative embodiments, be reversed without departing from the spirit and scope of the present disclosure. That is, alternative embodiments in which the lid 40 includes a notch and the platform 32 includes a latch are expressly contemplated herein. Furthermore, while the head 30 of the sample collection device 10 is primarily referred to in the context of including only a single latch and a single notch, it is appreciated that, in alternative embodiments, multiple latches and notches may be utilized without departing from the spirit and scope of the present disclosure.

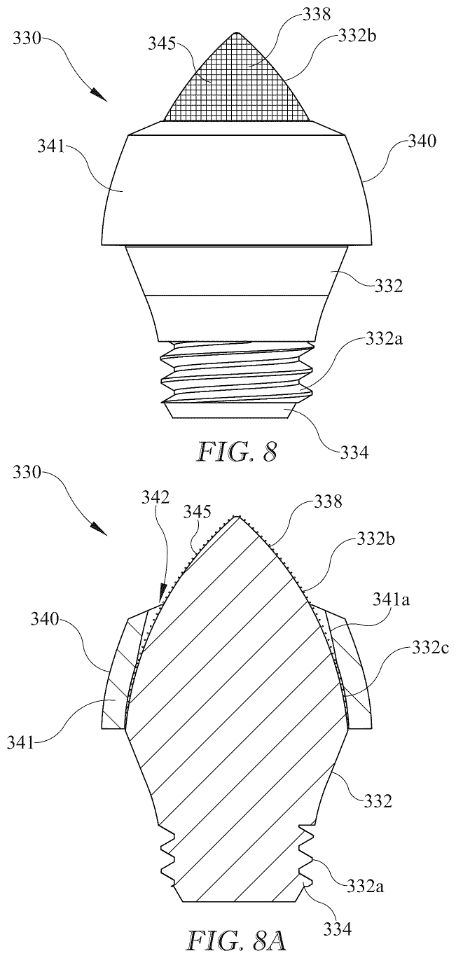

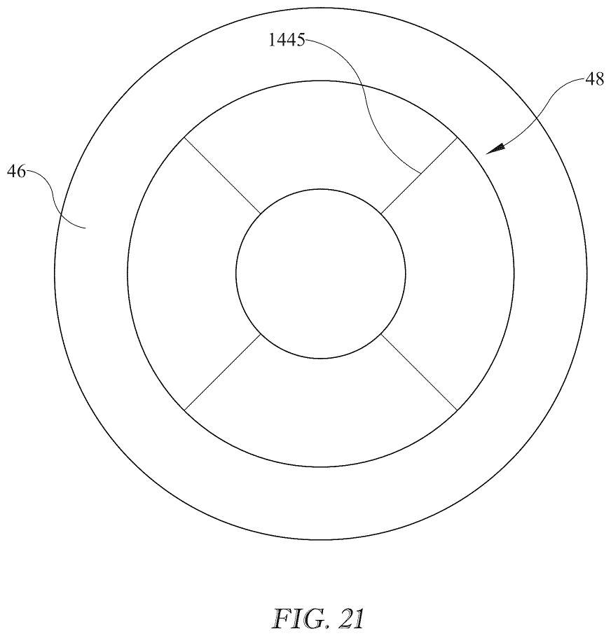

Referring now to FIGS. 2-5, 20, and 21, to permit direct contact between the sample card 50 and physical evidence when the sample collection device 10 is in use, the distal end 40b of the lid 40 includes a rim 46 which defines an opening 48. In this exemplary embodiment, the lid 40 also includes a permeable material 45 which is attached to the rim 46 and spans across the opening 48 to prevent the storage card 50 from falling through the opening 48 while still permitting direct contact between the storage card 50 and physical evidence when the sample collection device 10 is in use. In this exemplary embodiment, the permeable material 45 is a mesh with a square-like grid pattern that defines a plurality of openings 47. Of course, meshes with alternative grid pattern designs may also be employed. For instance, as shown in FIG. 21, in one alternative embodiment, a mesh 1445 with a grid pattern that defines a plurality of openings in which at least one of the openings is circular may be used to span the opening 48 defined by the rim 46. Alternative embodiments, in which the permeable material 45 is comprised of a fabric or other material suitable for preventing the storage card 50 from falling through the opening 48 while still permitting direct contact between the storage card 50 and physical evidence when the sample collection device 10 is in use are also contemplated. Additionally, and as further discussed below with reference to FIGS. 8, 8A, 9, 9A, 10, 10A, 11, and 12, embodiments in which no material spans the diameter of the opening defined by the rim of the lid are also contemplated herein.

Referring now again specifically to FIG. 5, sample cards which may serve as the sample card 50 and utilized with the exemplary sample collection devices disclosed herein include the Whatman® FTA classic card, the Copan® Nucleic-Card, and the Qiagen® FTA Elute card. Of course, other suitable nucleic acid storage cards that are designed for the collection, preservation, and shipment of biological samples for subsequent analysis may also be utilized without departing from the spirit and scope of the present disclosure. In some embodiments, the sample card 50 is designed to accommodate the collection of a single sample of physical evidence. In some embodiments, the sample card 50 is designed to accommodate multiple samples of physical evidence.

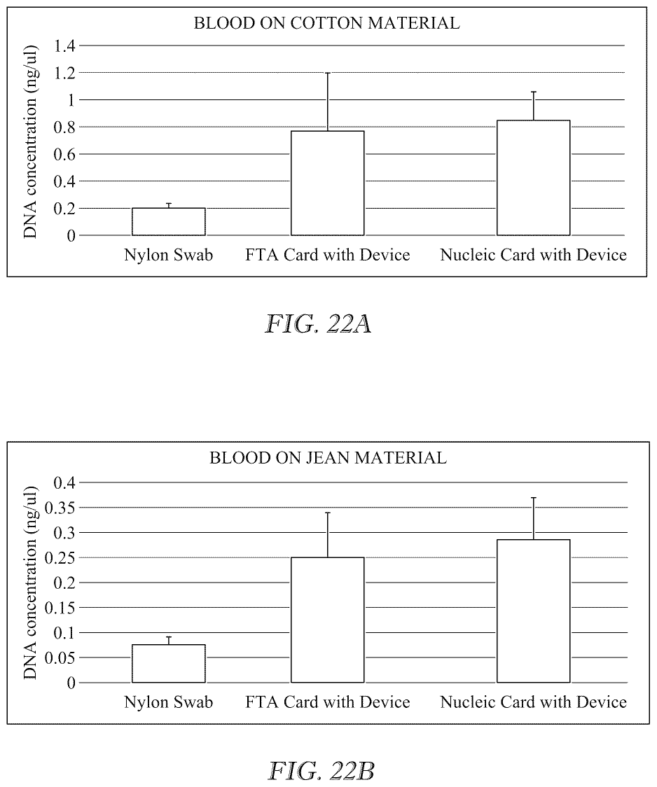

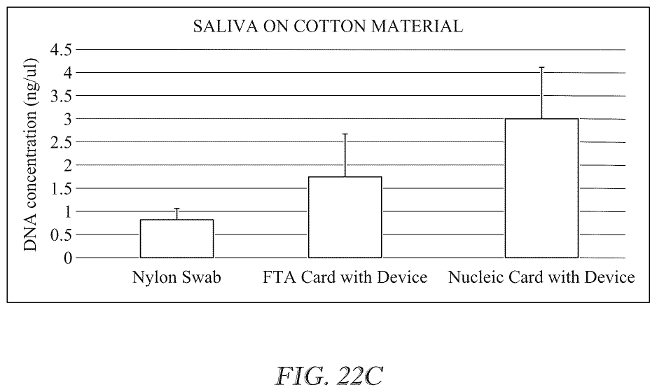

Referring now to FIGS. 22A, 22B, and 22C, to determine the efficacy of the sample collection device 10 loaded with a sample card 50 with respect to collecting samples of physical evidence as compared to traditional nylon swabs, samples of 10 μl blood provided on a cotton material, 10 μl blood provided on a jean material, and 10 μl saliva provided on a cotton material were collected utilizing a nylon swab, the sample collection device 10 loaded with a Whatman® FTA card, and the sample collection device 10 loaded with a Copan® Nucleic-Card. The samples collected were subsequently analyzed for DNA concentration using the Promega® Plexor® system. As shown in FIG. 22A, the DNA concentration within the blood samples collected from a cotton material using the sample collection device 10 loaded with the Whatman® FTA card and the sample collection device 10 loaded with the Copan® Nucleic-Card was approximately four times (4×) greater than the concentration of the sample collected using a traditional nylon swab. As shown in FIG. 22B, the DNA concentration within blood samples collected from a jean material using the sample collection device 10 loaded with the Whatman® FTA card and the sample collection device 10 loaded with the Copan® Nucleic-Card was approximately three and one-half times (3.5×) and four times (4×), respectively, greater than the concentration of the sample collected using a traditional nylon swab. As shown in FIG. 22C, the DNA concentration within the saliva samples collected from a cotton material using the sample collection device 10 loaded with the Whatman® FTA card and the sample collection device 10 loaded with the Copan® Nucleic-Card was approximately two times (2×) and four times (4×), respectively, greater than the concentration of the sample collected using a traditional nylon swab. The results of the foregoing tests thus suggest that the sample collection device 10 loaded with a sample card 50 was more effective in collecting higher concentrations of DNA from biological physical evidence than traditional nylon swabs.

Heads of alternative construction and/or with additional or alternative features relative to the head 30 described above with reference to FIGS. 2-5 can be utilized in combination with the handle 20 to form other exemplary sample collection devices made in accordance with the present disclosure.

Referring now to FIGS. 6A and 6B, another head 130 which may be utilized in combination with the handle 20, and in place of the head 30 described above with reference to FIGS. 2-5, to form another exemplary sample collection device made in accordance with the present disclosure is provided. In this embodiment, the head 130 also includes: a platform 132 with a proximal end 132a and a distal end 132b; and a lid 140 that is moveable relative to the platform 132, such that the lid 140 can be transitioned between a first (closed) position (FIG. 6A) to sandwich a card between the lid 140 and the platform 132 and a second (open) position (FIG. 6B) to permit deposit of a sample card onto, or removal of a sample card from, the platform 132. The proximal end 132a of the platform 132 is configured to be removably secured to the distal end 20a of the handle 20, and, in this regard, defines, and thus can be characterized as including, a threaded body 134 that is configured to be received in, and mate with the threading of, the threaded opening 26 defined by the distal end 20a of the handle 20 in the same manner as the threaded body 34 of the platform 32 described above with reference to FIGS. 2-5. The distal end 132b of the platform defines an upper surface 138 upon which a sample card 50 can be placed. In some embodiments, the diameter of the upper surface 38 of the platform 32 is between about one millimeter (1 mm) to about ten millimeters (10 mm). In some embodiments, the diameter of the upper surface 38 of the platform 32 is between about one millimeter (1 mm) and three millimeters (3 mm). In some embodiments, the diameter of the upper surface 38 of the platform 32 is between about three millimeters (3 mm) and four millimeters (4 mm). In some embodiments, the diameter of the upper surface 38 of the platform 32 is between about five millimeters (5 mm) and ten millimeters (10 mm). In this embodiment, the upper surface 138 of the platform 132 is a planar surface. Of course, the shape and/or diameter of the upper surface 138 can be modified to accommodate different types of physical evidence and/or environments in which physical evidence is located. In this embodiment, the platform 132 may be characterized as retaining a wedge-like shape.

Referring still to FIGS. 6A and 6B, like the lid 40 described above with reference to FIGS. 2-5, the lid 140 in this embodiment also includes a rim 144 that defines an opening 148 (as indicated by the area between the dashed lines in FIGS. 6A and 6B) which permits direct contact between a sample card provided on the upper surface of the platform 132 and physical evidence when the sample collection device of which the head 130 is a part is in use. Further like the lid 40 described above with reference to FIGS. 2-5, the lid 140 in this embodiment also includes a permeable material 145 consistent with that described above that is attached to the rim 144 and spans the diameter of the opening 148.

Referring still to FIGS. 6A and 6B, in this embodiment, the lid 140 is pivotally mounted to the platform 132 via a pivot 146, such that the lid 140 can pivot about a pivot axis defined by the pivot 146 to transition between the closed and open position. Accordingly, unlike the lid 40 and the platform 32 described above with reference to FIGS. 2-5, in this embodiment, the lid 140 is not fully, but, rather, only partially detached from the platform 132 when placed in the open position. In this embodiment, when the head 130 is secured to the handle 20 and the lid 140 is in the closed position, the longitudinal axis, l₁, of the handle 20 is orthogonal to the pivot axis defined by the pivot 146 and intersects with the lid 140.

Referring still to FIGS. 6A and 6B, to maintain the lid 140 in the closed position while the sample collection device of which the head 130 is a part is in use, in this embodiment, the lid 140 and the platform 132 are also configured to interlock with each other. In this regard, like the lid 40 and the platform 32 described above with reference to FIGS. 2-5, in this embodiment, the lid 140 and the platform 132 also include a latch 142 and a notch 136 that is configured to at least partially receive the latch 142, respectively. However, in this embodiment, the latch 142 and the notch 136 are not annularly shaped. As shown, in this embodiment, the lid 140 can be characterized as including a primary body 140a and an arm 140b. The primary body 140a of the lid 140 is sized to correspond to and cover the upper surface 138 defined by the distal end 132b of the platform 132. The arm 140b of the lid 140 defines the latch 142 and is connected to the primary body 140a in a manner which permits the arm 140b to move between: a first position (FIG. 6A) to deposit the latch 142 into the notch 136, thereby maintaining the lid 140 in the closed position while the sample collection device of which the head 130 is a part is in use; and a second position (FIG. 6B) to release the latch 142 from the notch 136, thereby permitting the lid 140 to be lifted to the open position.

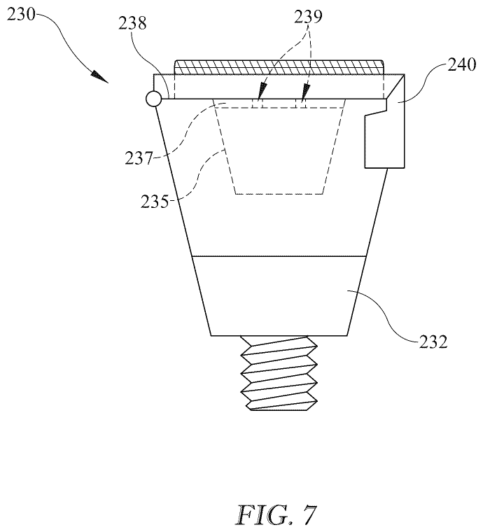

Referring now to FIG. 7, another head 230 which may be utilized in combination with the handle 20, and in place of the heads 30, 130 described above with reference to FIGS. 2-5, 6A, and 6B to form another exemplary sample collection device made in accordance with the present disclosure is provided. In this embodiment, the head 230 functions in the same manner as and is of identical construction to the head 130 described above with reference to FIGS. 6A and 6B, except as otherwise noted. In this regard, the head 230 in this embodiment also includes a platform 232 and a lid 240 which are secured to each other in the same manner as the platform 132 and the lid 140 described above with reference to FIGS. 6A and 6B. Unlike the platform 132 described above with reference to FIGS. 6A and 6B, however, in this embodiment, the platform 232 defines, and thus can be characterized as including, a reservoir 235 that is configured to hold a liquid (not shown) therein, such as a buffer solution. In this embodiment, the platform 232 also includes a film 237 or other pierceable cover which spans across an open end of the reservoir 235 to establish a physical barrier that prevents the liquid from inadvertently escaping the reservoir 235.

Referring still to FIG. 7, a user can manually pierce the film 237 to establish one or more ports 239 to permit the liquid to flow from the reservoir 235 to a sample card placed on the upper surface 238 defined by the distal end of the platform 232 by placing the head 230 in a tilted or inverted position while the lid 240 is in the closed position. Buffer solutions which may be utilized and provided in the reservoir 235 include, but are not necessarily limited to, forensic buffers designed to facilitate the extraction of DNA from biological samples. Suitable forensic buffers which may be utilized in the head 230 include, but not necessarily limited to the Prep-n-Go Buffer manufactured and distributed by Thermo Fisher Scientific of Waltham, Massachusetts, sterile PCR-grade water, and Tris-EDTA (TE) buffer. Thus, depending on the characteristic of the physical evidence to be collected, in this embodiment, a buffer solution can optionally be applied to a sample card in the above-described manner.

Although the one or more ports 239 permitting the flow of liquid to the sample card are referred to above as being established by virtue of a user manually piercing the film 237, alternative embodiments in which the one or more ports 239 are permanently defined as well as alternative embodiments in which the bottom of the lid 240 is provided with a spike or other implement configured to pierce the film 237 when the lid 240 is lowered are also contemplated herein. Furthermore, alternative embodiments in which the film 237 is removable are also contemplated herein. For instance, in an alternative embodiment, the film 237 may be comprised of a pull-tab film which can be pulled to disassociate the film 237 from the platform 232 to permit liquid to flow from the reservoir 235 to the a sample card placed on the upper surface 238 are also contemplated herein.

Referring now to FIGS. 8 and 8A, another head 330 which may be utilized in combination with the handle 20, and in place of the heads 30, 130, 230 described above with reference to FIGS. 2-5, 6A, 6B, and 7 to form another exemplary sample collection device made in accordance with the present disclosure is provided. In this embodiment, the head 330 also includes: a platform 332 with a proximal end 332a and a distal end 332b; and a lid 340 that is moveable relative to the platform 332, such that the lid 340 can be transitioned between a first (closed) position (FIGS. 8 and 8A) and a second (open) position (not shown) to permit deposit of a sample card onto, or removal of the sample card from, the platform 332. The proximal end 332a of the platform 332 is configured to be removably secured to the distal end 20a of the handle 20, and, in this regard, defines, and thus can be characterized as including, a threaded body 334 that is configured to be received in, and mate with the threading of, the threaded opening 26 defined by the distal end 20a of the handle 20 in the same manner as the threaded body 34 of the platform 32 described above with reference to FIGS. 2-5. The distal end 332b of the platform 332 defines an upper surface 338 of the platform 332 upon which a sample card can be placed. As shown, in this embodiment, the distal end 332b of the platform 332 is shaped such that the upper surface 338 of the platform 332 retains a rounded spear-like shape. Accordingly, in this embodiment, the diameter of the platform 332 increases as the platform 332 extends from its distal end 332b to a midportion 332c of the platform 332. The foregoing construction may thus, in some instances, permit the platform 332 to be held perpendicular to surfaces on which physical evidence is deposited and to which traditional swabs cannot typically be held perpendicular to. In some embodiments, the diameter of the upper surface 338 of the platform 332 is between about five millimeters (5 mm) and about 15 millimeters (15 mm). Of course, the diameter of the upper surface 338 of the platform can be adjusted to better accommodate different types of physical evidence or environments in which physical evidence is located.

Referring still to FIGS. 8 and 8A, in this embodiment, the lid 340 includes an annular body 341 and a permeable material 345 consistent with that of the heads 30, 130, 230 described above with reference to FIGS. 2-5, 6A, 6B, and 7. The annular body 341 defines a central opening 342 through which the distal end 332b of the platform 332 is inserted through to transition the lid 340 to the closed position. The permeable material 345 is sized to cover the upper surface 338 of the platform 332 and to cover a sample card deposited on the upper surface 338 when the lid 340 is placed in the closed position. In this embodiment, the lid 340 receives a portion of the platform 332 and a portion of the platform 332 extends through the central opening 342 when the lid 340 is in the closed position.

As shown in FIG. 8A, an interior surface 341a of the annular body 341 defining the central opening 342 corresponds to an exterior surface defining the midportion 332c of the platform 332, such that the midportion 332c of the platform 332 cannot be inserted entirely through the central opening 342. The corresponding interior surface 341a of the annular body 341 and the exterior surface defining the midportion 332c of the platform 332 thus permit the lid 340 to be maintained in the closed position via a friction fit connection (which also may be referred to as a press fit connection or an interface fit connection) when the outer perimeter of the permeable material 345 is positioned between the interior surface 341a of the annular body 341 and the exterior surface defining the midportion 332c of the platform 332 and the platform 332 is fully inserted into the central opening 342 of the lid 340. Accordingly, when a sample card is loaded onto the upper surface 338 of the platform 332 and the lid 340 is placed in the closed position, the sample card will be sandwiched between the permeable material 345 of the lid 340 and the platform 332, and, potentially, depending on the size of the sample card, the annular body 341 of the lid 340 and the platform 332. In this embodiment, the interior surface 341a of the annular body 341 of the lid 340 is tapered, such that the space between an first portion the interior surface 341a of the annular body 341 and the exterior surface defining the midportion 332c of the platform 332 can accommodate the thickness of the permeable material 345 and the sample card, and the space between a second, lower portion of the interior surface 341a of the annular body 341 and the exterior surface defining the midportion 332c of the platform 332 can only accommodate the permeable material 345.

Referring now again to FIGS. 8 and 8A, embodiments in which the permeable material 345 is secured to the annular body 341 as well as embodiments in which the permeable material 345 is not secured to the annular body 341 are contemplated herein. With respect to the former, the outer perimeter of the permeable material 345 can be secured to the interior surface 341a of the annular body 341 using known joining techniques (e.g., using adhesive), thus permitting the annular bod 341 and the permeable material 345 to be moved as a single unit and placed in association with the platform 332 at the same time. With respect to the latter, after depositing the sample card on the upper surface 338 of the platform 332, the permeable material 345 would first be positioned over the sample card and the distal end 332b of the platform 332 would then be fully inserted through the central opening 342 defined by the annular body 341 to place and maintain the lid 340 in the closed position.

Referring still to FIGS. 8 and 8A, the platform 332 is considered to be fully inserted into the central opening 342 of the lid 340 when the platform 332 is inserted through the central opening 342 to the point where the interior surface 341a of the annular body 341 defining the central opening 342 and the exterior surface defining the midportion 332c of the platform 332 prevent further insertion of the platform 332. To transition the lid 340 to the open position, the lid 340 is lifted to a point where the platform 332 is no longer fully inserted into the central opening 342 and the sample card can be deposited onto or removed from the upper surface 338 of the platform 332. Like the lid 40 described above with reference to FIGS. 2-5, in this embodiment, the lid 340 can be completely removed from engagement with the platform 332.

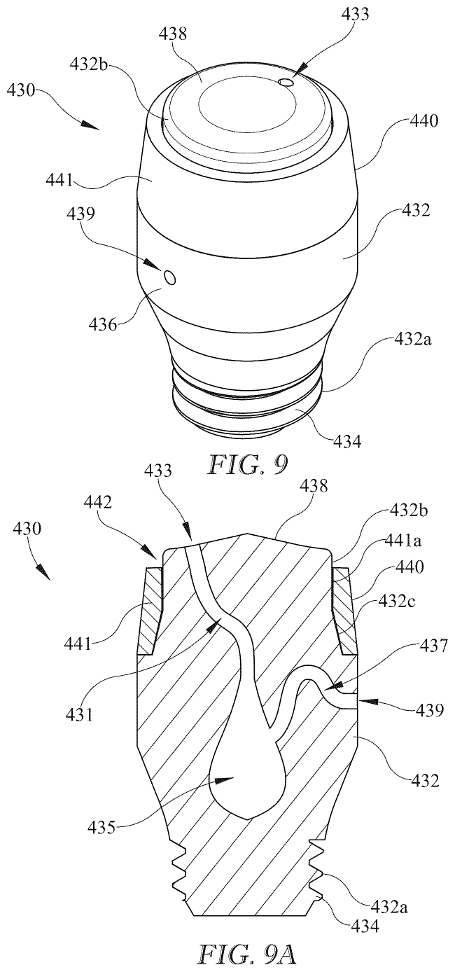

Referring now to FIGS. 9 and 9A, another head 430 which may be utilized in combination with the handle 20, and in place of the heads 30, 130, 230, 330 described above with reference to FIGS. 2-5, 6A, 6B, 7, 8, and 8A to form another exemplary sample collection device made in accordance with the present disclosure is provided. In this embodiment, the head 430 also includes: a platform 432 with a proximal end 432a and a distal end 432b; and a lid 440 that is moveable relative to the platform 432, such that the lid 440 can be transitioned between a first (closed) position (FIGS. 9 and 9A) and a second (open) position (not shown) to permit deposit of a sample card onto, or removal of the sample card from, the platform 432. The proximal end 432a of the platform 432 is configured to be removably secured to the distal end 20a of the handle 20, and, in this regard, defines, and thus can be characterized as including, a threaded body 434 that is configured to be received in, and mate with the threading of, the threaded opening 26 defined by the distal end 20a of the handle 20 in the same manner as the threaded body 34 of the platform 32 described above with reference to FIGS. 2-5. The distal end 432b of the platform 432 defines an upper surface 438 of the platform 432 upon which a sample card can be placed. As shown, in this embodiment, the upper surface 438 of the platform 432 is circular and gradually slopes downwardly as the upper surface 438 extends from its center to its outer perimeter. Without wishing to be bound by any particular theory, the foregoing platform design is believed to be particularly well-suited for collecting dry samples of physical evidence. In some embodiments, the diameter of the upper surface 438 may be between about five millimeters (5 mm) an about 10 millimeters (10 mm). Of course, the shape and/or diameter of the upper surface 438 can be modified to accommodate different types of physical evidence and/or environments in which physical evidence is located.

Referring still to FIGS. 9 and 9A, like the head 230 described above with reference to FIG. 7, in this embodiment, the platform 432 also includes a reservoir 435 configured to hold a liquid, such as a buffer solution, and one or more ports 431, 437 which are in fluid communication with the reservoir 435. Specifically, in this embodiment, there are two ports 431, 437 in fluid communication with the reservoir 435: a first port 431 which terminates at, and is in fluid communication with, an opening 433 defined by the upper surface 438 of the platform 432; and a second port 437 which terminates at, and is in fluid communication with, an opening 439 defined by an exterior side surface 436 of the platform. Unlike the head 230 described above with reference to FIG. 7, in this embodiment, the ports 431, 437 and the openings 433, 439 are permanently defined in the platform 432, as opposed to being established by virtue of a user piercing a film. In use, the first port 431 provides a pathway for liquid within the reservoir 435 to travel into contact with a sample card deposited on the upper surface 438 of the platform 432 when the head 430 is tilted or inverted. The second port 437 can be utilized to deposit the liquid into the reservoir 435. In this regard, to deposit the liquid into the reservoir 435, the platform 432 can be turned so that the opening 439 defined by the exterior side surface 436 of the platform 432 is facing upward, inserting a syringe or other suitable injection device loaded with the liquid into the opening 439 defined by the exterior side surface 436, and injecting the liquid into the second port 437. The foregoing reservoir 435, port 431, 437, and opening 433, 439 arrangement may serve to reduce contamination risk as compared to applying liquid directly to a sample card provided on the upper surface of the platform 432 (e.g., by permitting an external water source to be directed through the interior of platform 432). In some embodiments a film, such as a pull-tab film, may be applied over the openings 433, 439 prior to use of the head 430.

Referring still to FIGS. 9 and 9A, like the lid 340 described above with reference to FIGS. 8 and 8A, in this embodiment, the lid 440 also includes an annular body 441 and a permeable material (not shown) consistent with that of the heads 30, 130, 230, 330 described above with reference to FIGS. 2-5, 6A, 6B, 7, 8, and 8A. In FIGS. 9 and 9A, the permeable material of the lid 440 has intentionally been omitted to better show certain features of the platform 432. The annular body 441 defines a central opening 442 through which the distal end 432b of the platform 432 is inserted through to transition the lid 440 to the closed position. The permeable material is sized to cover the upper surface 438 of the platform 432 and to cover a sample card deposited on the upper surface 438 when the lid 440 is placed in the closed position. In this embodiment, the lid 440 receives a portion of the platform 432 and a portion of the platform 432 extends through the central opening 442 when the lid 440 is in the closed position.

As shown in FIG. 9A, an interior surface 441a of the annular body 441 defining the central opening 442 corresponds to an exterior surface defining a midportion 432c of the platform 432, such that the midportion 432c of the platform 432 cannot be inserted entirely through the central opening 442. The corresponding interior surface 441a of the annular body 441 and the exterior surface defining the midportion 432c of the platform 332 thus permit the lid 440 to be maintained in the closed position via a friction fit connection when the outer perimeter of the permeable material is positioned between the interior surface 441a of the annular body 441 and the exterior surface defining the midportion 432c of the platform 432 and the platform 432 is fully inserted into the central opening 442 of the lid 440. Accordingly, when a sample card is loaded onto the upper surface 438 of the platform 432 and the lid 440 is placed in the closed position, the sample card will be sandwiched between the permeable material of the lid 440 and the platform 432, and, potentially, depending on the size of the sample card, the annular body 441 of the lid 440 and the platform 432.

Referring now again to FIGS. 9 and 9A, embodiments in which the permeable material is secured to the annular body 441 as well as embodiments in which the permeable material is not secured to the annular body 441 are contemplated herein. With respect to the former, the outer perimeter of the permeable material can be secured to the interior surface 441a of the annular body 441 using known joining techniques (e.g., using adhesive), thus permitting the annular bod 441 and the permeable material to be moved as a single unit and placed in association with the platform 432 at the same time. With respect to the latter, after depositing the sample card on the upper surface 438 of the platform 432, the permeable material would first be positioned over the sample card and the distal end 432b of the platform 432 would then be fully inserted through the central opening 442 defined by the annular body 441 to place and maintain the lid 440 in the closed position.

Referring still to FIGS. 9 and 9A, the platform 432 is considered to be fully inserted into the central opening 442 of the lid 440 when the platform 432 is inserted through the central opening 442 to the point where the interior surface 441a of the annular body 341 defining the central opening 442 and the exterior surface defining the midportion 432c of the platform 432 prevent further insertion of the platform 432. To transition the lid 440 to the open position, the lid 440 is lifted to a point where the platform 432 is no longer fully inserted into the central opening 442 and the sample card can be deposited onto or removed from the upper surface 438 of the platform 432. Like the lid 40 described above with reference to FIGS. 2-5, in this embodiment, the lid 440 can be completely removed from engagement with the platform 432.

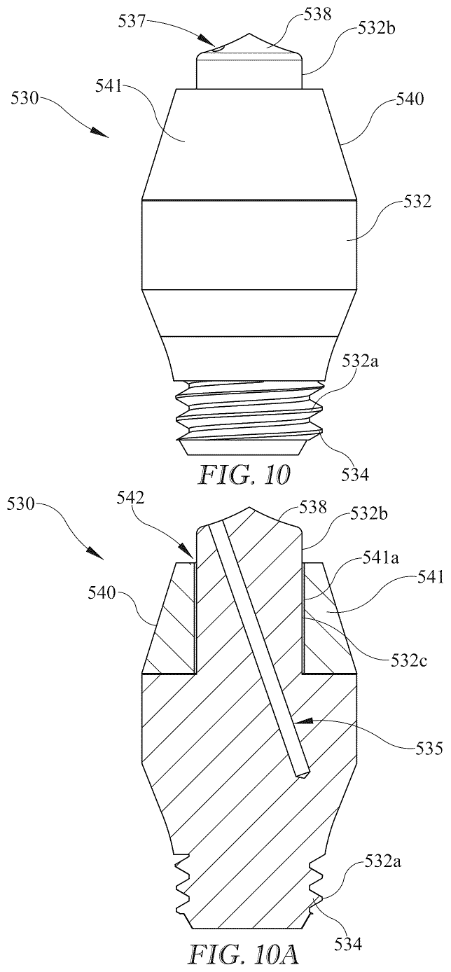

Referring now to FIGS. 10 and 10A, another head 530 which may be utilized in combination with the handle 20, and in place of the heads 30, 130, 230, 330, 430 described above with reference to FIGS. 2-5, 6A, 6B, 7, 8, 8A, 9, and 9A to form another exemplary sample collection device made in accordance with the present disclosure is provided. In this embodiment, the head 530 also includes: a platform 532 with a proximal end 532a and a distal end 532b; and a lid 540 that is moveable relative to the platform 532, such that the lid 440 can be transitioned between a first (closed) position (FIGS. 10 and 10A) and a second (open) position (not shown) to permit deposit of a sample card onto, or removal of the sample card from, the platform 532. The proximal end 532a of the platform 532 is configured to be removably secured to the distal end 20a of the handle 20, and, in this regard, defines, and thus can be characterized as including, a threaded body 534 that is configured to be received in, and mate with the threading of, the threaded opening 26 defined by the distal end 20a of the handle 20 in the same manner as the threaded body 34 of the platform 32 described above with reference to FIGS. 2-5. The distal end 532b of the platform 532 defines an upper surface 538 of the platform 532 upon which a sample card can be placed. As shown, in this embodiment, the upper surface 538 of the platform 532 is circular and gradually slopes downwardly at a steeper incline as the upper surface 538 extends from its center to its outer perimeter as compared to the upper surface 438 of the platform 432 described above with reference to FIGS. 9 and 9A. Without wishing to be bound by any particular theory, the foregoing platform design is believed to be particularly well-suited for collecting small, low volumes and/or dry samples of physical evidence. In this embodiment, the diameter of the upper surface 538 is from about five millimeters (5 mm) to about ten millimeters (10 mm). Of course, the shape and/or diameter of the upper surface 538 can be modified to accommodate different types of physical evidence and/or environments in which physical evidence is located.

Referring still to FIGS. 10 and 10A, like the heads 230, 430 described above with reference to FIGS. 7, 9, and 9A, in this embodiment, the platform 532 also includes a reservoir 535 that is configured to hold a liquid consistent with that described above. However, in this embodiment, the reservoir 535 is comprised of a single channel which terminates at, and is in fluid communication with, an opening 537 defined by the upper surface 538 of the platform 532. In this embodiment, the channel defining the reservoir 535 and the opening 537 defined by the upper surface 538 of the platform 532 are permanently defined in the platform 532, as opposed to being established by virtue of a user piercing a film. In this embodiment, the opening 537 is positioned adjacent to the perimeter of the upper surface 538 and the channel defining reservoir 535 diagonally extends from the opening 537 within the interior of the platform 532. In use, liquid can be deposited into the reservoir 535 via a syringe or other suitable injection device and then subsequently applied to a sample card deposited on the upper surface 538 of the platform 532 by tilting or inverting the platform 532. Without wishing to be bound by any particular theory, the foregoing reservoir 535 and opening 537 design is believed to reduce the risk of contamination when moistening a sample card provided on the upper surface 538 of the platform 532 as compared to using an external tool (e.g., a pipette) to apply liquid to the sample card. In some embodiments a film, such as a pull-tab film, may be applied over the opening 537 prior to use of the head 530.

Referring still to FIGS. 10 and 10A, like the lids 340, 440 described above with reference to FIGS. 8, 8A, 9, and 9A, in this embodiment, the lid 540 also includes an annular body 541 and a permeable material (not shown) consistent with that of the heads 30, 130, 230, 330, 430 described above with reference to FIGS. 2-5, 6A, 6B, 7, 8, 8A, 9, and 9A. In FIGS. 10 and 10A, the permeable material of the lid 540 has intentionally been omitted to better show certain features of the platform 532. The annular body 541 defines a central opening 542 through which the distal end 532b of the platform 532 is inserted through to transition the lid 540 to the closed position. The permeable material is sized to cover the upper surface 538 of the platform 532 and to cover a sample card deposited on the upper surface 538 when the lid 540 is placed in the closed position. In this embodiment, the lid 540 receives a portion of the platform 532 and a portion of the platform 532 extends through the central opening 542 when the lid 540 is in the closed position.

As shown in FIG. 10A, an interior surface 541a of the annular body 541 defining the central opening 542 corresponds to an exterior surface defining the midportion 532c of the platform 532. The corresponding interior surface 541a of the annular body 541 and the exterior surface defining the midportion 532c of the platform 532 thus permit the lid 540 to be maintained in the closed position via a friction fit connection when the outer perimeter of the permeable material of the lid 540 is positioned between the interior surface 541a of the annular body 541 and the exterior surface defining the midportion 532c of the platform 532 and the platform 532 is fully inserted into the central opening 542 of the lid 540. Accordingly, when a sample card is loaded onto the upper surface 538 of the platform 532 and the lid 540 is placed in the closed position, the sample card will be sandwiched between the permeable material of the lid 540 and the platform 532, and, potentially, depending on the size of the sample card, the annular body 541 of the lid 540 and the platform 532.

Referring now again to FIGS. 10 and 10A, embodiments in which the permeable material of the lid 540 is secured to the annular body 541 as well as embodiments in which the permeable material is not secured to the annular body 541 are contemplated herein. With respect to the former, the outer perimeter of the permeable material can be secured to the interior surface 541a of the annular body 541 using known joining techniques (e.g., using adhesive), thus permitting the annular bod 541 and the permeable material to be moved as a single unit and placed in association with the platform 532 at the same time. With respect to the latter, after depositing the sample card on the upper surface 538 of the platform 532, the permeable material would first be positioned over the sample card and the distal end 532b of the platform 532 would then be fully inserted through the central opening 542 defined by the annular body 541 to place and maintain the lid 540 in the closed position.

Referring still to FIGS. 10 and 10A, the platform 532 is considered to be fully inserted into the central opening 542 of the lid 540 when the platform 532 is inserted through the central opening 542 to the point where the platform 532 cannot be further inserted. As shown, in this embodiment, the platform 532 defines a shelf which extends perpendicular to the interior surface 541a of the annular body 541 and establishes a barrier which stops the platform 532 from being further inserted through the central opening 542. To transition the lid 540 to the open position, the lid 540 is lifted to a point where the platform 532 is no longer fully inserted into the central opening 542 and the sample card can be deposited onto or removed from the upper surface 538 of the platform 532. Like the lid 40 described above with reference to FIGS. 2-5, in this embodiment, the lid 540 can be completely removed from engagement with the platform 532.

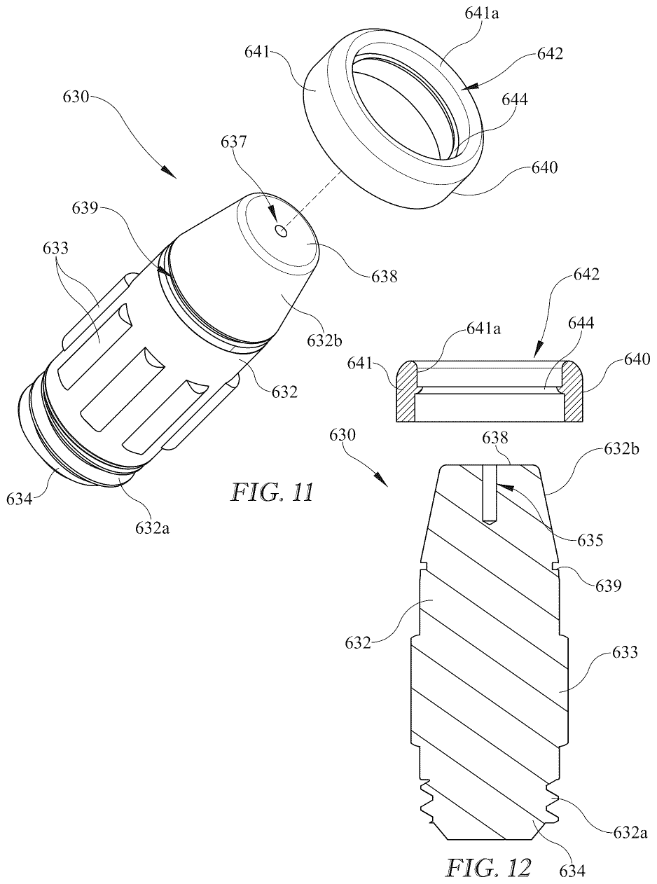

Referring now to FIGS. 11 and 12, another head 630 which may be utilized in combination with the handle 20, and in place of the heads 30, 130, 230, 330, 430, 530 described above with reference to FIGS. 2-5, 6A, 6B, 7, 8, 8A, 9, 9A, 10, and 10A to form another exemplary sample collection device made in accordance with the present disclosure is provided. In this embodiment, the head 630 also includes: a platform 632 with a proximal end 632a and a distal end 632b; and a lid 640 that is moveable relative to the platform 632, such that the lid 640 can be transitioned between a first (closed) position (not shown) and a second (open) position (FIGS. 11 and 12) to permit deposit of a sample card onto, or removal of the sample card from, the platform 632. The proximal end 532a of the platform 532 is configured to be removably secured to the distal end 20a of the handle 20, and, in this regard, defines, and thus can be characterized as including, a threaded body 634 that is configured to be received in, and mate with the threading of, the threaded opening 26 defined by the distal end 20a of the handle 20 in the same manner as the threaded body 34 of the platform 32 described above with reference to FIGS. 2-5. The distal end 632b of the platform 632 defines an upper surface 638 of the platform 632 upon which a sample card can be placed. As shown, in this embodiment, the upper surface 638 of the platform 632 is a planar surface. In this embodiment, the diameter of the upper surface 638 is from about five millimeters (5 mm) to about ten millimeters (10 mm). Of course, the shape and/or diameter of the upper surface 638 can be modified to accommodate different types of physical evidence and/or environments in which physical evidence is located. As further shown, in this embodiment, the exterior surface of the midportion of the platform 632 defines a plurality of ribs 633 positioned about the circumference of the midportion of the platform 632. In use, the plurality of ribs 633 provide a series of grips which a user can engage to rotate the platform 632 in either a first rotational direction to associate the head 630 with the handle 20 or a second rotational direction to disassociate the head 630 from the handle 20.

Referring still to FIGS. 11 and 12, like the head 530 described above with reference to FIGS. 10 and 10A, in this embodiment, the platform 632 also includes a reservoir 635 that is comprised of a single channel which terminates at an opening 637 defined by the upper surface 638 of the platform 632. In this embodiment, the channel defining the reservoir 635 and the opening 637 defined by the upper surface 638 of the platform 632 are permanently defined in the platform 632, as opposed to being established by virtue of a user piercing a film. In this embodiment, the opening 637 is centrally positioned about the upper surface 638 of the platform 632 and the channel defining the reservoir 635 extends perpendicular to the upper surface 638 toward the proximal end 632a of the platform 632, as shown in FIG. 12. In use, liquid can be deposited into the reservoir 635 via a syringe or other suitable injection device and then subsequently applied to a sample card deposited on the upper surface 638 of the platform 632 by tilting or inverting the platform 632. The central positioning of the reservoir 635 and opening 637 permits liquid within the reservoir 635 to be evenly spread about a sample card deposited on the upper surface 338 when the head 630 is inverted. In some embodiments a film, such as a pull-tab film, may be applied over the opening 637 prior to use of the head 630.

Referring still to FIGS. 11, and 12, like the lids 340, 440, 540 described above with reference to FIGS. 8, 8A, 9, 9A, 10, and 10A, in this embodiment, the lid 640 also includes an annular body 641 and a permeable material (not shown) consistent with that of the heads 30, 130, 230, 330, 430, 530 described above with reference to FIGS. 2-5, 6A, 6B, 7, 8, 8A, 9, 9A, 10, and 10A. In FIGS. 11 and 12, the permeable material of the lid 640 has intentionally been omitted to better show certain features of the platform 632. The annular body 641 defines a central opening 642 through which the distal end 632b of the platform 632 is inserted through to transition the lid 640 to the closed position. The permeable material is sized to cover the upper surface 638 of the platform 632 and to cover a sample card deposited on the upper surface 638 when the lid 640 is placed in the closed position. In this embodiment, the lid 640 receives a portion of the platform 632 and a portion of the platform 632 extends through the central opening 642 when the lid 640 is in the closed position.

Referring still to FIGS. 11 and 12, unlike the lids 340, 440, 540 described above with reference to FIGS. 8, 8A, 9, 9A, 10, and 10A, the lid 640 in this embodiment is not maintained in the closed position via a friction fit connection. Rather, in this embodiment, like the lid 40 of the head 30 described above with reference to FIGS. 2-5, the lid 640 includes an annular latch 644, which, in this case, is defined by the annular body 641 of the lid 640, and the platform includes a corresponding annular notch 639 that is sized to at least partially receive the latch 644 therein. Accordingly, to transition the lid 640 to the closed position and interlock the lid 640 and the platform 632 together, the distal end 632b of the platform 632 is inserted through the central opening 642 defined by the lid 640 until the latch 644 is deposited in the notch 639. In this embodiment, when the head 630 is loaded with a sample card and the lid 640 is placed in the closed position, the sample card will be sandwiched between the permeable material of the lid 640 and the platform 632, and, potentially, depending on the size of the sample card, the annular body 641 of the lid 640 and the platform 632.

Referring still to FIGS. 11 and 12, embodiments in which the permeable material of the lid 640 is secured to the annular body 641 as well as embodiments in which the permeable material is not secured to the annular body 641 are contemplated herein. With respect to the former, the outer perimeter of the permeable material can be secured to an interior surface 641a of the annular body 641 using known joining techniques (e.g., using adhesive), thus permitting the annular bod 641 and the permeable material to be moved as a single unit and placed in association with the platform 632 at the same time. With respect to the latter, after depositing the sample card on the upper surface 638 of the platform 632, the permeable material would first be positioned over the sample card and the distal end 632b of the platform 632 would then be fully inserted through the central opening 642 defined by the annular body 641 to place and maintain the lid 640 in the closed position. In this embodiment, the interior surface 641a of the annular body 641 of the lid 640 may be characterized as being tapered, such that the space between a first portion of the interior surface 641a extending from the top of the annular body 641 to the latch 644 and an exterior surface of the platform 632 can accommodate the thickness of the permeable material and a sample card, and the space between the latch 644 and the notch 639 of the platform 632 can only accommodate the permeable material of the lid 640.

Referring still to FIGS. 11 and 12, to transition the lid 640 to the open position, a user can pull the lid 640 while holding the handle 20 or proximal end 632a of the platform 632 until the latch 644 is released from the notch 639 and the platform 632 and the lid 640 are completely separated. Thus, like the lids 40, 340, 440, 540 described above with reference to FIGS. 2-5, 8, 8A, 9, 9A, 10, and 10A, in this embodiment, the lid 640 can be completely removed from engagement with the platform 632.

Although the lid 640 and the platform 632 are primarily referred to herein as including the latch 644 and the notch 639, respectively, it is appreciated that the locations of the latch 644 and the notch 639 can, in alternative embodiments, be reversed without departing from the spirit and scope of the present disclosure.

Although the lids 340, 440, 540, 640 described above with reference to FIGS. 8, 8A, 9, 9A, 10, 10A, 11, and 12 are primarily referred to in the context of including both an annular body 341, 441, 541, 641 and a permeable material 345, alternative embodiments in which such lids 340, 440, 540, 640 are comprised of the annular body 341, 441, 541, 641 alone (i.e., without the permeable material) are also contemplated herein. Such alternative embodiments may be utilized in instances where the sample card has a diameter and thickness which permits the sample card to be sandwiched between the annular body 341, 441, 541, 641 of the lid 340, 440, 540, 640 and the platform 332, 432, 532, 632 to which the lid 340, 440, 540, 640 corresponds, and, in the case of lids 340, 440, 540, permit such lids 340, 440, 540 to be maintained in the closed position via a friction fit connection when placed in the closed position while the sample card is loaded on the platform 332, 432, 5332 corresponding to such lids 340, 440, 540.

Platforms of alternative construction relative to those of the heads 30, 130, 230, 330, 430, 530, 630 described above with reference to FIGS. 2-5, 6A, 6B, 7, 8, 8A, 9, 9A, 10, 10A, 11, and 12 can be utilized in alternative head constructions and in combination with the handle 20 to form other exemplary sample collection devices made in accordance with the present disclosure.

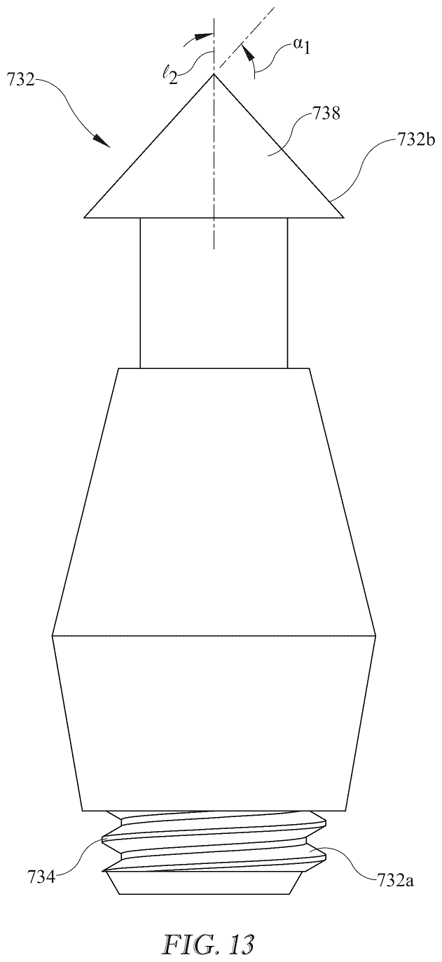

Referring now to FIG. 13, another platform 732 which may be utilized in an alternative head construction is provided. The platform 732 includes a proximal end 732a that is configured to be removably secured to the distal end 20a of the handle 20, and, in this regard, defines, and thus can be characterized as including, a threaded body 734 that is configured to be received in, and mate with the threading of, the threaded opening 26 defined by the distal end 20a of the handle 20 in the same manner as the threaded body 34 of the platform 32 described above with reference to FIGS. 2-5. The platform 732 also includes a distal end 732b that defines an upper surface 738 of the platform 732 upon which a sample card can be placed. In this embodiment, the diameter of the upper surface 738 of the platform is about 12 millimeters (12 mm). Of course, the diameter of the upper surface 738 can be modified to accommodate different types of physical evidence and/or environments in which physical evidence is deposited. In this embodiment, the upper surface 738 of the platform 732 is a conical surface which forms an angle, α₁, with respect to the longitudinal axis, l₂, of the platform 732. The particular angle, α₁, exhibited by the conical upper surface 738 can be adapted to better accommodate different types of physical evidence and/or crime scene environments. Like the platform 332 described above with reference to FIGS. 8 and 8A, the foregoing construction may thus, in some instances, permit the platform 732 to be held perpendicular to surfaces on which physical evidence is deposited and to which traditional swabs cannot typically be held perpendicular to. In some embodiments, the angle, α₁, ranges from about 20 degrees (20°) to about 60 degrees (60°). Although not shown, it is appreciated that, in some embodiments implementations, the platform 732 may be utilized in combination with a lid to maintain a sample card deposited on the upper surface 738 of the platform in position while a sample collection device of which the platform 732 is a part is in use. For instance, in some embodiments and implementations, the platform 732 may be utilized in combination with a lid with an annular body which defines a central opening and is maintained in association with the platform 732 via a friction fit connection in similar fashion as the lids 340, 440, 540 described above with reference to FIGS. 8, 8A, 9, 9A, 10, and 10A. In some embodiments and implementations, the platform 732 alone may define the head of a sample collection device to which the platform is a part of. In some implementations, a sample card deposited on the upper surface 738 of the platform 732 may be maintained in position by taping a portion of the sample card to a portion of the platform 732 or via other suitable securement means.

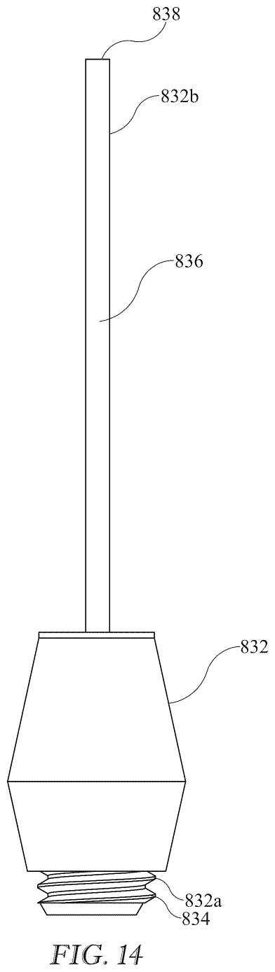

Referring now to FIG. 14, another platform 832 which may be utilized in an alternative head construction is provided. The platform 832 includes a proximal end 832a that is configured to be removably secured to the distal end 20a of the handle 20, and, in this regard, defines, and thus can be characterized as including, a threaded body 834 that is configured to be received in, and mate with the threading of, the threaded opening 26 defined by the distal end 20a of the handle 20 in the same manner as the threaded body 34 of the platform 32 described above with reference to FIGS. 2-5. The platform 832 also includes a distal end 832b that defines an upper surface 838 of the platform 832 upon which a sample card can be placed. As shown, in this embodiment, the distal end 832b of the platform 832 is defined by an elongated cylindrical body 836 and the upper surface 838 is a planar surface. In this embodiment, the upper surface 838 of the platform 832 is about 1.2 millimeters (1.2 mm) in diameter and the cylindrical body 836 defining the distal end 832b of the platform 832 is about two centimeters (2 cm) to about ten centimeters (10 cm) in length. Accordingly, the platform 832 may prove useful in collecting physical evidence deposited within cavities with narrow passages and/or small deposits of physical evidence. Of course, the shape of the upper surface 838, diameter of the upper surface 838, and/or length of the cylindrical body 836 may be adjusted to accommodate different types of physical evidence and/or crime scene environments. Although not shown, it is appreciated that, in some embodiments and implementations, the platform 832 may be utilized in combination with a lid to maintain a sample card deposited on the upper surface 838 of the platform 832 in position while a sample collection device of which the platform 832 is a part is in use. In some embodiments and implementations, the platform 832 alone may define the head of a sample collection device to which the platform is a part of. In some embodiments and implementations, a sample card deposited on the upper surface 838 of the platform 832 may be maintained in position by taping a portion of the sample card to a portion of the platform 832 or via other suitable securement means.

As reflected in FIGS. 15-19, a variety of other platforms 932, 1032, 1132, 1232, 1332, of different shapes and/or dimension as compared to the platforms 32, 132, 232, 332, 432, 532, 832 described above with reference to FIGS. 2-5, 6A, 6B, 7, 8, 9, 9A, 10, 10A, 11, and 12, may also be utilized in alternative head constructions and in combination with the handle 20 to form other exemplary sample collection devices made in accordance with the present disclosure. Each respective platform 932, 1032, 1132, 1232, 1332 shown in FIGS. 15-19 includes a proximal end 932a, 1032a, 1132a, 1232a, 1332a that is configured to removably be secured to the distal end 20a of the handle 20, and, in this regard, defines, and thus can be characterized as including, a threaded body 934, 1034, 1134, 1234, 1334 that is configured to be received in, and mate with the threading of, the threaded opening 26 defined by the distal end 20a of the handle 20 in the same manner as the threaded body 34 of the platform 32 described above with reference to FIGS. 2-5. Each respective platform 932, 1032, 1132, 1232, 1332 shown in FIGS. 15-19 also includes a distal end 932b, 1032b, 1132b, 1232b, 1332b that defines an upper surface 938, 1038, 1138, 1238, 1338 of the platform 932, 1032, 1132, 1232, 1332 upon which a sample card can be placed.

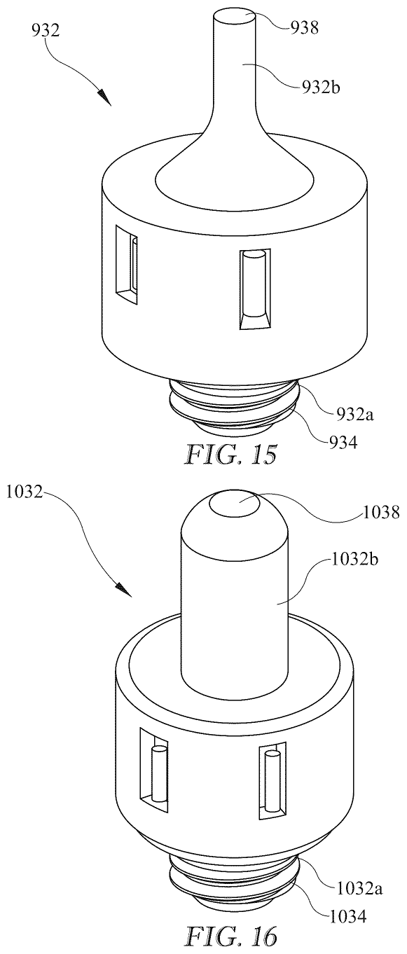

As evidenced by viewing FIGS. 15-19 the respective platforms 932, 1032, 1132, 1232, 1332 in FIGS. 15-19, the dimensions of the upper surfaces 938, 1038, 1138, 1238, 1338 vary across the different platforms 932, 1032, 1132, 1232, 1332 to accommodate the collection of a single sample or multiple samples of physical evidence. Specifically, in the embodiment of the platform 932 shown in FIG. 15, the upper surface 938 of the platform 932 has a diameter of about one millimeter (1 mm) to about three millimeters (3 mm) and is configured to support a sample card that is large enough to collect approximately one sample of physical evidence for subsequent analysis. In the embodiment of the platform 1032 shown in FIG. 16, the upper surface 1038 of the platform 1032 has a diameter of about three millimeters (3 mm) to about four millimeters (4 mm) and is configured to support a sample card that is large enough to collect about three to about five samples of physical evidence for subsequent analysis. In the embodiments of the platforms 1132, 1232, 1332 shown in FIGS. 17-19, the upper surfaces 1138, 1238, 1338 of the platforms 1132, 1232, 1332 have a diameter ranging from about five millimeters (5 mm) to about ten millimeters (10 mm) and are configured to support a sample cart that is large enough to collect about nine to about fourteen samples of physical evidence for subsequent analysis. In some embodiments and implementations, each respective platform 932, 1032, 1132, 1232, 1332 alone may define the head of a sample collection device to which the platform is a part of. In some embodiments and implementations, a sample card deposited on the upper surface 938, 1038, 1138, 1238, 1338 may be maintained in position by taping a portion of the sample card to a portion of the platform 932, 1032, 1132, 1232, 1332 or via other suitable securement means.

The handles, platforms, and lids of the exemplary sample collection devices described herein can be fully or partially constructed of a plastic material or combination of plastic materials to reduce the overall weight exhibited by the sample collection devices and thus strain or fatigue incurred by a user while using the sample collection devices. Non-limiting examples of plastics which may be utilized in the construction of the exemplary sample collection device include polylactic acid (PLA) plastic, polyethylene terephthalate glycol (PETG) plastic, carbon fiber reinforced PLA plastic, polypropylene plastic, and nylon. Of course other suitable materials may alternatively be utilized in the construction of the handles, platforms, and/or lids without departing from the spirt and scope of the present disclosure. In this regard, alternative embodiments in which the handles, platforms, and/or lids of the exemplary sample collection devices disclosed herein are fully or partially constructed of an alternative plastic, a metal, wood, and/or glass material are also contemplated herein.

It is appreciated that while, the proximal end 32a, 132a, 232a, 332a, 432a, 532a, 632a, 732a, 832a, 932a, 1032a, 1132a, 1232a, 1332a of the platforms 32, 132, 232, 332, 432, 532, 632, 732, 832, 932, 1032, 1132, 1232, 1332 described above with reference to FIGS. 2-5, 6A, 6B, 7, 8, 8A, 9, 9A, 10, 10A, 11-19 are primarily referred to as defining, and thus including, a threaded body and the distal end 20a of the handle 20 is primarily referred to as defining, and thus including, a threaded opening for receiving the threaded body that, in alternative embodiments, the proximal end 32a, 132a, 232a, 332a, 432a, 532a, 632a, 732a, 832a, 932a, 1032a, 1132a, 1232a, 1332a of the platforms 32, 132, 232, 332, 432, 532, 632, 732, 832, 932, 1032, 1132, 1232, 1332 may include the threaded opening and the distal end 20a of the handle 20 may include the threaded body without departing from the spirit and scope of the present disclosure. Additionally, although the handle 20 is primarily referred to herein as being removably secured to the platforms 32, 132, 232, 332, 432, 532, 632, 732, 832, 932, 1032, 1132, 1232, 1332 described above with reference to FIGS. 2-5, 6A, 6B, 7, 8, 8A, 9, 9A, 10, 10A, 11-19, alternative embodiments in which the handle 20 is permanently secured to the platforms 32, 132, 232, 332, 432, 532, 632, 732, 832, 932, 1032, 1132, 1232, 1332 are also contemplated herein. Furthermore, while the reservoirs referred to herein are primarily identified in the context of being included in a platform of the sample collection device, alternative embodiments in which the reservoir is included in the handle 20 are also contemplated herein. In such alternative embodiments, the platform of the sample collection device would include one or more ports to place the reservoir in fluid communication with the upper surface of the platform of the sample collection device.

In some embodiments, a sample card may form part of the exemplary sample collection devices described herein. That is, in some embodiments, the exemplary sample collection devices described herein may further include a sample card.

The presently disclosed subject matter also includes a kit for collecting a forensic evidence.

An exemplary kit for collecting a forensic evidence made in accordance with the present disclosure includes: one of the exemplary sample collection devices described herein; and a sample card consistent with that described above. In some embodiments, the kit may further include a buffer solution for dampening the sample card while secured to the sample collection device. Buffer solutions which may be utilized in the kit include, but are not necessarily limited to, forensic buffers designed to facilitate the extraction of DNA from biological samples. For instance, suitable buffer solutions which may be included in the kit are the Prep-n-Go Buffer manufactured and distributed by Thermo Fisher Scientific of Waltham, Massachusetts, sterile PCR-grade water, and/or Tris-EDTA (TE) buffer. In some embodiments, the kit may further include a pipette for transferring the buffer solution onto the sample card. A variety of pipettes suitable for such application are readily known and available in the art. In some embodiments, the kit may further include instructions which specify how to properly use the sample collection device, the sample card, and buffer solution and/or how to properly store the sample card once the forensic sample is collected.

The presently disclosed subject matter also includes a method for collecting and processing forensic evidence.

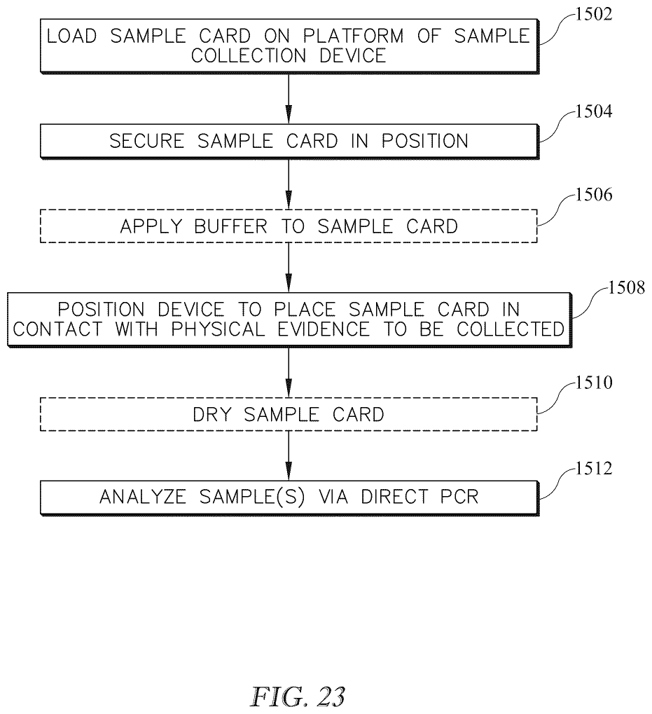

Referring now to FIG. 23, an exemplary method for collecting and processing forensic evidence commences by loading a sample card configured to collect one or more samples when placed into contact with physical evidence onto a sample collection device made in accordance with the present disclosure, as indicated in block 1502. In some implementations, loading the sample card onto the platform of the sample collection device involves transitioning the lid of the sample collection device from a closed position to an open position, if the lid is not already in the open position, and depositing the sample card onto the upper surface of the platform of the sample collection device. Once the sample card is deposited on the upper surface of the sample collection device, the sample card is then secured in position on the platform, as indicated by block 1504. In some implementations, the sample card is secured in position by transitioning the lid of the sample collection device from the open position to the closed position so that the sample card is sandwiched between the lid and the platform of the sample collection device. In instances where the physical evidence to be collected is biological material which includes DNA, such as saliva, blood, mucus, semen, urine, and the like, the use of a buffer solution designed to facilitate the extraction of DNA from a biological material may prove beneficial. Accordingly, in some implementations, the exemplary method may further include applying a buffer solution to the sample card, as indicated in block 1506. Both implementations in which the application of the buffer solution to the sample card is performed prior to sample collection as well as implementations in which the application of the buffer solution is performed subsequent to sample collection (e.g., after one or more samples on the sample card have dried) are contemplated herein.

Referring still to FIG. 23, once the sample card is secured in place and treated, if necessary or desired, with a buffer solution, the sample collection device is positioned as to place the sample card into contact with the physical evidence to collect one or more samples of the physical evidence on the sample card. In some implementations, one or more biological samples are collected by positioning the sample collection device as to place the sample card into physical contact with a biological material. In instances where the physical evidence collected is wet material, such as saliva, blood, mucus, semen, urine, etc., it may prove beneficial to dry the collected samples prior to processing and analyzing the sample. Accordingly, in some implementations, the exemplary method further includes drying the one or more samples collected on the sample card, as indicated by block 1510. In some implementations, the one or more samples collected may be dried by virtue of being allowed to sit and air dry. In some implementations, the one or more samples collected may be dried in a suitable heating cabinet. Once the one or more samples collected are dried, if necessary or desired, at least one of the samples collected on the sample card is analyzed by subjecting the sample or samples to a direct PCR protocol, as indicated by block 1512. Direct PCR protocols which may be employed to analyze collected samples include, but are not limited to those disclosed in Hall D E, Roy R. An evaluation of direct PCR amplification. Croat Med J. 2014 December; 55(6):655-61. doi: 10.3325/cmj.2014.55.655. PMID: 25559837; PMCID: PMC4295066.

Referring still to FIG. 23, in some implementations, the exemplary method may further include punching out or otherwise removing one or more portions of the sample card on which a collected sample is deposited prior to subjecting the sample or samples to the direct PCR protocol. In some implementations, subsequent to the one or more samples being collected on the sample card, the sample card may be stored for a predetermined period of time while secured on the platform of the sample collection device prior to analysis via direct PCR. In one such implementation, the platform on which the sample card is provided may be disassociated from the handle of the sample collection device prior to storage. In cases where the sample card utilized is large enough to hold multiple collected samples, analyzing the collected samples may involve subjecting different samples collected on the sample card to a direct PCR protocol at different times. For instance, in some implementations, a first sample collected on the sample card may be subject to a direct PCR protocol at a first time, and a second sample collected on the sample card may be subjected to a direct PCR protocol at a second, later time. In some implementations, the same direct PCR protocol may be utilized across different samples. In some implementations, different direct PCR protocols may be utilized across different samples. In some implementations, the exemplary method takes less than about 3 hours to complete.

Although the exemplary sample collection devices, kits, and methods disclosed herein may sometimes be referred to as being utilized to collect and/or process biological samples, it should be appreciated that the exemplary sample collection devices may also find utility with respect to the collection of other types of physical evidence, such as soil or clothing.

One of skill in the art will recognize that additional embodiments and implementations are also possible without departing from the teachings of the present disclosure. This detailed description, and particularly the specific details of the exemplary embodiments and implementations disclosed therein, is given primarily for clarity of understanding, and no unnecessary limitations are to be understood therefrom, for modifications will become obvious to those skilled in the art upon reading this disclosure and may be made without departing from the spirit or scope of the disclosure.

What is claimed is:

1. A sample collection device, comprising:
a handle; and
a head secured to a distal end of the handle and configured to receive and maintain a sample card for collecting a sample at a distal end of the sample collection device, the head including
a platform with a proximal end and a distal end, the proximal end of the platform secured to the handle, and the distal end of the platform defining an upper surface for receiving the sample card; and
a lid moveable relative to the platform, such that the lid can be transitioned between (i) a first position to sandwich the sample card between the lid and the platform and (ii) a second position to permit deposit of the sample card onto or removal of the sample card from the upper surface of the platform.

2. The sample collection device of claim 1, wherein the lid is configured to receive at least a portion of the platform when the lid is placed in the first position.

3. The sample collection device of claim 2, wherein the lid includes a cavity in which the distal end of the platform is deposited when the lid is placed in the first position.

4. The sample collection device of claim 1, wherein the lid includes an opening.

5. The sample collection device of claim 4, wherein a portion of the platform extends through the opening when the lid is in the first position.

6. The sample collection device of claim 4, wherein the lid includes a permeable material that spans across the opening.

7. The sample collection device of claim 6, wherein the permeable material defines a plurality of openings.

8. The sample collection device of claim 7, wherein the permeable material is a mesh.

9. The sample collection device of claim 1, wherein the platform is removably secured to the handle.

10. The sample collection device of claim 9, wherein one of the proximal end of the platform and a distal end of the handle includes a threaded body and the other of the proximal end of the platform and the distal end of the handle includes a threaded opening for receiving the threaded body.

11. The sample collection device of claim 1, wherein the lid is removably secured to the platform, such that the lid can be completely removed from engagement with the platform to transition the lid to the second position.

12. The sample collection device of claim 1, wherein the lid is pivotally mounted to the platform.

13. The sample collection device of claim 1, wherein one of the platform and the lid includes one or more latches and the other of the platform and the lid includes one or more notches, and wherein the one or more latches are at least partially received within the one or more notches when the lid is in the first position.

14. The sample collection device of claim 1, wherein the lid is maintained in the first position by a friction fit connection when the head is loaded with the sample card and the lid is placed in the first position.

15. The sample collection device of claim 1, wherein the platform includes a reservoir configured to receive a liquid.

16. The sample collection device of claim 15, wherein the reservoir is in fluid communication with an opening defined by the upper surface of the platform.

17. The sample collection device of claim 16, wherein the reservoir is in fluid communication with an opening defined by an exterior side surface of the platform.

18. The sample collection device of claim 1, wherein the upper surface of the platform is a planar surface.

19. The sample collection device of claim 1, wherein the upper surface of the platform forms an angle ranging between about 20 degrees to about 60 degrees with respect to a longitudinal axis of the platform.

20. The sample collection device of claim 1, wherein the handle includes a plurality of depressions and a plurality of protrusions, and wherein the handle is symmetrical about a longitudinal axis of the handle.

21. A kit for collecting a forensic evidence, comprising:
a sample card configured to collect one or more samples when placed into contact with physical evidence; and
a sample collection device, including
a handle, and
a head secured to a distal end of the handle and configured to receive and maintain the sample card at a distal end of the sample collection device, the head including
a platform with a proximal end and a distal end, the proximal end of the platform secured to the distal end of the handle, and the distal end of the platform defining an upper surface for receiving the sample card, and
a lid moveable relative to the platform.

22. The kit of claim 21, and further comprising a buffer solution.

23. A method for collecting and processing forensic evidence, comprising:
loading a sample card for collecting one or more samples onto an upper surface of a platform of a sample collection device;
securing the sample card in position on the upper surface of the platform by sandwiching the sample card between the platform and a lid of the sample collection device; and
positioning the sample collection device so that the sample card is placed into contact with a biological material to collect one or more biological samples; and
subjecting at least one of the one or more collected biological samples to a direct polymerase chain reaction (PCR) protocol.
